(19) United States
(12) Patent Application Publication
(10) Pub. No.: US 2007/0105839 A1
(43) Pub. Date: May 10, 2007

(54) 2, 4-Di (phenylamino) pyrimidines useful in the treatment of proliferative disorders

(76) Inventors: Patricia Imbach, Kaiseraugst (CH); Johannes Roesel, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/571,733

(22) PCT Filed: Sep. 17, 2004
(86) PCT No.: PCT/EP04/10466

§ 371 Date: Mar. 14, 2006

Related U.S. Application Data
(60) Provisional application No. 60/504,374, filed on Sep. 18, 2003.

(51) Int. Cl. A61K 31/55; A61K 31/541; A61K 31/5377; A61K 31/506; A61K 31/505

(52) U.S. Cl. 514/217.05; 514/275; 514/235.5; 514/227.5; 514/252.14

(57) ABSTRACT

A method of preventing or treating proliferative disorders such as a tumor disease, by inhibiting ALK activity with compounds of formula (1):

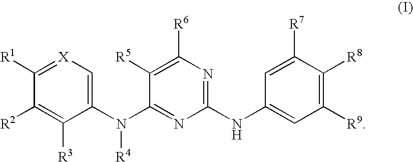

The present invention relates the use of pyrimidine derivatives for the treatment of proliferative disorders, such as cancer, and to pharmaceutical compositions comprising them for the treatment of such proliferative disorders.

More particularly the present invention is based on the discovery that certain pyrimidine derivatives possess valuable, pharmacologically useful properties. In particular the pyrimidine derivatives used according to the present invention exhibit specific inhibitory activities that are of pharmacological interest. They are effective especially as protein tyrosine kinase inhibitors; they exhibit, for example, powerful inhibition of the tyrosine kinase activity of anaplastic lymphoma kinase (ALK) and the fusion protein of NPM-ALK .This protein tyrosine kinase results from a gene fusion of nucleophosmin (NPM) and the anaplastic lymphoma kinase (ALK), rendering the protein tyrosine kinase activity of ALK ligand-independent. NPM-ALK plays a key role in signal transmission in a number of hematopoetic and other human cells leading to hematological and neoplastic diseases, for example in anaplastic large-cell lymphoma (ALCL) and non-Hodgkin's lymphomas (NHL), specifically in ALK+NHL or Alkomas, in inflammatory myofibroblastic tumors (IMT) and neuroblastomas. In addition to NPM-ALK other gene fusions have been identified in human hematological and neoplastic diseases; mainly TPM3-ALK (a fusion of nonmuscle tropomyosin with ALK). The pyrimidine derivatives are useful for the inhibition of all such ALK-containing gene fusions.

The compounds that are useful as inhibitors of ALK or a gene fusion containing ALK are especially compounds of formula I 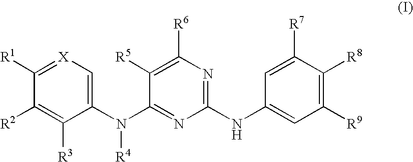
wherein
X is ═CR⁰— or ═N—;
each of R⁰, R¹, R², R³ and R⁴ independently is hydrogen; hydroxy; C₁-C₈alkyl; C₂-C₈alkenyl; C₃-C₈cycloalkyl; C₃-C₈cycloalkyl-C₁-C₈alkyl; hydroxyC₁-C₈alkyl; C₁-C₈alkoxyC₁-C₈alkyl; hydroxyC₁-C₈alkoxyC₁-C₈alkyl; arylC₁-C₈alkyl which optionally may be substituted on the ring by hydroxy, C₁-C₈alkoxy, carboxy or C₁-C₈alkoxycarbonyl;
or R³ and R⁴ form together with the nitrogen and carbon atoms to which they are attached a 5 to 10 membered heterocyclic ring and comprising additionally 1, 2 or 3 heteroatoms selected from N, O and S;
or each of R¹, R² and R³, independently, is halogen; halo-C₁-C₈alkyl; C₁-C₈alkoxy; halo-C₁-C₈alkoxy; hydroxyC₁-C₈alkoxy; C₁-C₈alkoxyC₁-C₈alkoxy; aryl; arylC₁-C₈alkoxy; heteroaryl; heteroaryl-C₁-C₄alkyl; 5 to 10 membered heterocyclic ring; nitro; carboxy; C₂C₈alkoxycarbonyl; C₂-C₈alkylcarbonyl; —N(C₁-C₈alkyl)C(O) C₁-C₈alkyl; —N(R¹⁰)R¹¹; —CON(R¹⁰)R¹¹; —SO₂N(R¹⁰)R¹¹; or —C₁-C₄-alkylene—SO₂N(R¹⁰)R¹¹; wherein each of R¹⁰ and R¹¹ independently is hydrogen; hydroxy; C₁-C₈alkyl; C₂-C₈alkenyl; C₃-C₈cycloalkyl; C₃-C₈cycloalkyl-C₁-C₈alkyl; C₁-C₈alkoxyC₁-C₈alkyl; hydroxyC₁-C₈alkoxyC₁-₈alkyl; hydroxyC₁-C₈alkyl; (C₁-C₈alkyl)-carbonyl; arylC₁-C₈alkyl which optionally may be substituted on the ring by hydroxy, C₁-C₈alkoxy, carboxy or C₂-C₈alkoxycarbonyl; or 5 to 10 membered heterocyclic ring;
or R¹ and R² form together with the C-atoms to which they are attached aryl or a 5 to 10 membered heteroaryl residue comprising one or two heteroatoms selected from N, O and S; or
each of R⁵ and R⁶ independently is hydrogen; halogen; cyano; C₁-C₈alkyl; halo-C₁-C₈alkyl; C₂-C₈alkenyl; C₂-C₈alkynyl; C₃-C₈cycloalkyl; C₃-C₈cycloalkylC₁-C₈alkyl; C₅-C₁₀arylC₁-C₈alkyl;
each of R⁷, R⁸ and R⁹ is independently hydrogen; hydroxy; C₁-C₈alkyl; C₂-C₈alkenyl; halo-C₁-₈alkyl; C₁-C₈alkoxy; C₃-C₈cycloalkyl; C₃-C₈cycloalkylC₁-C₈alkyl; arylC₁-C₈alkyl; —Y—R¹² wherein Y is a direct bond or O and R¹² is a substituted or unsubstituted 5, 6 or 7 membered heterocyclic ring comprising 1, 2 or 3 heteroatoms selected from N, O and S; carboxy; (C₁-C₈alkoxy)-carbonyl; —N(C₁₋₈alkyl)—CO—NR¹⁰R¹¹; —CONR¹⁰R¹¹; —N(R¹⁰)(R¹); —SO₂N(R¹⁰)R¹¹; or R⁷ and R⁸ or R⁸ and R⁹, respectively form together with the carbon atoms to which they are attached, a 5 or 6 membered heteroaryl comprising 1, 2 or 3 heteroatoms selected from N, O and S; or a 5 or 6 membered carbocyclic ring. in free form or salt form.

Any aryl may be phenyl, naphthyl or 1,2,3,4-tetrahydronaphthyl, preferably phenyl. Heteroaryl is an aromatic heterocyclic ring, e.g. a 5 or 6 membered aromatic heterocyclic ring, optionally condensed to 1 or 2 benzene rings and/or to a further heterocylic ring.

Any heterocyclic ring may be saturated or unsaturated and optionally condensed to 1 or 2 benzene rings and/or to a further heterocyclic ring.

Examples of heterocyclic rings or heteroaryl include e.g. morpholinyl, piperazinyl, piperidyl, pyrrolidinyl, pyridyl, purinyl, pyrimidinyl, N-methyl-aza-cycloheptan-4yl, indolyl, quinolinyl, isoquinolinyl, 1,2,3,4-tetrahydroquinolinyl, benzothiazolyl, thiazolyl, imidazolyl, benzimidazolyl, benzoxadiazolyl, benzotriazolyl, indanyl, oxadiazolyl, pyrazolyl, triazolyl, and tetrazolyl. Preferred heterocyclic rings or heteroaryl are morpholinyl, piperazinyl, piperidyl, pyrrolidinyl, pyridyl, N-methyl-aza-cycloheptan-4-yl, thiazolyl, imidazolyl and tetrazolyl.

When R⁷ and R⁸ or R³ and R⁹ form together with the carbon atoms to which they are attached a 5 or 6 membered carbocyclic ring, this may preferably be cyclopentyl or cyclohexyl.

Halo-alkyl is alkyl wherein one or more H are replaced by halogen, e.g. CF₃.

Any alkyl or alkyl moiety may be linear or branched. C₁₋₈alkyl is preferably C₁₋₄alkyl. C₁₋₈ alkoxy is preferably C₁₋₄alkoxy. Any alkyl, alkoxy, alkenyl, cycloalkyl, heterocyclic ring, aryl or heteroaryl may be, unless otherwise stated, unsubstituted or substituted by one or more substituents selected from halogen; OH; C₁-C₈alkyl; C₁-C₈alkoxy; nitro; cyano; COOH; carbamoyl; C(NH₂)═NOH; —N(R¹⁰)R¹¹; C₃-C₆cycloalkyl; 3 to 7 membered heterocyclic ring; phenyl; phenyl-C₁₋₄alkyl; 5 or 6 membered heteroaryl. When alkyl, alkoxy or alkenyl is substituted, the substituent is preferably on the terminal C atom. When the heterocyclic ring or heteroaryl is substituted, e.g. as disclosed above, this may be on one or more ring carbon atoms and/or ring nitrogen atom when present. Examples of a substituent on a ring nitrogen atom are e.g.

C₁₋₈alkyl, carbamoyl, —C(NH₂)═NOH, —NR¹⁰R¹¹, C₃₋₆cycloalkyl or phenyl-C₁₋₄alkyl, preferably C₁₋₈alkyl, C₃₋₆cycloalkyl or phenyl-C₁₋₄alkyl.

Preferably substituted alkyl or alkoxy as R₇ is alkyl or alkoxy substituted on the terminal C atom by OH, C₁₋₄alkoxy or a heterocyclic ring. When R¹⁰ or R¹¹ is a 5 to 10 membered heterocyclic ring, it may be e.g. thiazolyl.

Halogen may be F, Cl, Br, or l.

Preferably at most one of R¹, R² or R³ is CONR¹⁰R¹¹ or SO₂NR¹⁰R¹¹ more preferably SO₂NR¹⁰R¹¹.

The compounds of the invention may exist in free form or in salt form, e.g. addition salts with e.g. organic or inorganic acids, for example trifluoroacetic acid or hydrochloride acid, or salts obtainable when they comprise a carboxy group, e.g. with a base, for example alkali salts such as sodium, potassium, or substituted or unsubstituted ammonium salts.

In formula I the following significances are preferred independently, collectively or in any combination or sub-combination:
(a) X is ═CR⁰;
(b) R⁰ is hydrogen; halogen, e.g. Cl; C₁-C₄alkyl, e.g. methyl or ethyl; C₁₋₄alkoxy, e.g. methoxy; preferably hydrogen;
(c) R¹ is hydrogen; halogen, e.g. Cl or F; OH; C₁-C₈alkyl, e.g. methyl or ethyl; substituted C₁₋₈alkyl, e.g. terminally OH substituted C₁₋₈alkyl; —SO₂N(R¹⁰)R¹¹; —N(C₁₋₄alkyl)C(O) C₁₋₄ alkyl; a 5 or 6 membered heterocydic ring optionally substituted on a ring N atom (when possible); C₁-C₈alkoxy, e.g. methoxy; aryl, e.g. phenyl; or form together with R² and the C-atoms to which R¹ and R² are attached 5 to 10 membered aryl or heteroaryl, the latter comprising 1 or 2 nitrogen atoms;
(d) R² is hydrogen; hydroxy; C₁-C₈alkyl, e.g. methyl or ethyl; substituted C₁₋₈alkyl, e.g. terminally OH—or C₁₋₄-alkoxy substituted C₁₋₈alkyl; C₁₋₈alkoxy; C₁-C₄alkoxyC₁-C₈alkoxy; —CON(R¹⁰)R¹¹; —SO₂N(R¹⁰)R¹¹; or forms together with R¹ and the C-atoms to which R¹ and R² are attached a 5 to 10 membered aryl or heteroaryl, the latter comprising 1 or 2 nitrogen atoms;
(e) R³ Is hydrogen; halogen, e.g. Cl, Br; hydroxy; C₁-C₈alkyl, e.g. methyl or ethyl; substituted C₁₋₈alkyl, e.g. terminally OH substituted C₁₋₈alkyl; carboxy; CONR¹⁰R¹¹; —SO₂N(R¹⁰)R¹¹; a 5 or 6 membered heterocyclic ring optionally substituted on a ring nitrogen atom (when possible); or forms together with R⁴ and the N and C atoms to which R³ and R⁴ are attached a 6 membered heterocyclic ring;
(f) R⁴ is hydrogen; or forms together with R³ and the N and C atoms to which R³ and R⁴ are attached a 6 membered heterocyclic ring; preferably hydrogen;
(g) R⁵ is hydrogen; halogen; C₁₋₄alkyl; or CF₃;
(h) R⁶ is hydrogen;
(i) R⁷ is hydrogen; hydroxy; C₁₋₄alkyl; substituted C₁₋₄alkyl, e.g. terminally OH substituted C₁₋₄alkyl; C₁₋₈alkoxy; substituted C₁₋₈alkoxy, e.g. terminally substituted by OH, C₁₋₄alkoxy or a heterocyclic ring; NR¹⁰R¹¹; —SO₂N(R¹⁰)R¹¹; —Y—R¹²; CF₃; or R⁷forms together with R⁸ and the C-atoms to which R⁷ and R⁸ are attached a 5 membered heteroaryl residue, e.g. bridged by —NH—CH═CH—, —CH═CH—NH—, —NH—N═CH—, —CH═N—NH—, —NH—N═N— or —N═N—NH—;
(k) R⁸ is hydrogen; hydroxy; C₁₋₄alkoxy; carboxy; a 5 or 6 membered heterocyclic ring optionally substituted on a ring C or N atom; N(C₁₋₄alkyl)—CO— NR¹⁰R¹¹; or forms with R⁷ or R⁹ and the C-atoms to which R⁷ and R⁸ or R⁸ and R⁹ , respectively, are attached a 5 membered heteroaryl residue, e.g. bridged by —NH—CH═CH—, —CH═CH—NH—, —NH—N═CH—, —CH═N—NH—, —NH—N═N— or —N═N—NH—;
(I) R⁹ is hydrogen; C₁₋₄alkoxy; NR¹⁰R¹¹; or forms with R⁸ and the C atoms to which R⁸ and R⁹ are attached a 5 membered heteroaryl, e.g. bridged by —NH—CH═CH—, —CH═CH—NH—, —NH—N═CH—, —CH═N—NH—, —NH—N═N— or—N═N—NH—;
(m) one of R¹⁰ and R¹¹, independently, is hydrogen or C₁₋₄alkyl and the other is hydrogen; OH; C₁₋₈alkyl, substituted C₁₋₈alkyl, e.g. terminally substituted by OH, C₃₋₆cycloalkyl or a heterocyclic ring; C₂₋₈alkenyl; C₃₋₈cycloalkyl; hydroxyC₁₋₈alkoxyC₁₋₈alkyl; or a 5 membered heterocyclic ring.
R³ is preferably SO₂NR¹⁰R¹¹.

The invention also provides the use of a compound of formula I for the preparation of a medicament for the treatment of a hematological and neoplastic disease.

The present invention also provides a process for the production of a compound of formula I, comprising reacting a compound of formula II 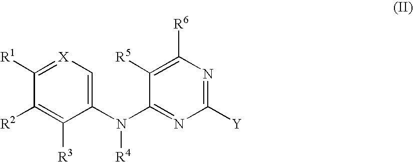
wherein R¹, R², R³, R⁴, R⁵, R⁶ and X are as defined above, and Y is a leaving group, preferably halogen such as bromide, iodine, or in particular chloride;
with a compound of formula III 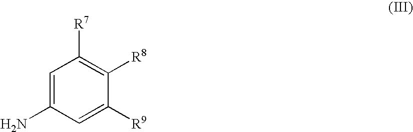
wherein R⁷, R⁸ and R⁹ are as defined above;
and recovering the resulting compound of formula I in free or in form of a salt, and, where required, converting the compound of formula I obtained in free form into the desired salt form, or vice versa.

The process may be performed according to methods known in the art, e.g. as described in examples 1 to 4.

The compound of formula II used as starting materials may be obtained by reacting a compound of formula IV 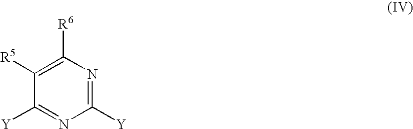
with a compound of formula V 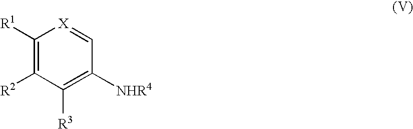
wherein R¹, R², R³, R⁴, R⁵, R⁸, Y and X are as defined above.

The compounds of formula IV and V are known or may be produced in accordance with known procedures.

The following examples illustrate the invention without any limitation.

The following abbreviations are employed: APC=allophycocyanine, BINAP=2,2′-bis(diphenylphosphino)-1,1′-binaphthyl, cDNA=complementary DNA, DCM=dichloromethane, DIAD=diisopropyl azodicarboxylate, DMAP=4-dimethylaminopyridine, DMF=dimethylformamide, DMSO=dimethylsulfoxide, DMF=dimethylformamide; Pmc=2,2,5,7,8pentamethylchroman; tBu=tert.-butyl; DIPCDI=N,N′-diisopropylcarbodiimid; DTT=1,4-dithio-D,L-treitol, DNA=deoxyribonucleic acid, EDTA=ethylenediaminetetra-acetic acid, Lck=lymphoid T-cell protein tyrosine kinase, LAT-11=linker for activation of T cell, RT=room temperature; RT-PCR=reverse transcription polymerase chain reaction, MS=molecular ion (e.g. M+H¹⁺) determined by electrospray mass spectroscopy; Eu=europium.

### EXAMPLE 1

### 2-[2-(1H-Indazol-6-ylamino)-pyrimidin-4-ylamino]-benzenesulfonamide

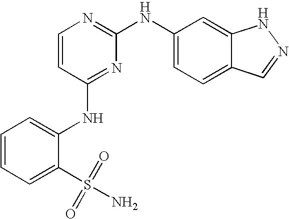

(a) 2-(2-Chloro-pynmidin-4-ylamino)-benzenesulfonamide: To a suspension of 8.52 g (49.47 mmol) 2-aminobenzenesulfonamide in 200 ml isopropanol is added 22.1 g (148.42 mmol, 3 equivalent) 2,4-dichloropyrimidine and 20 ml 10 M hydrochloric acid (200 mmol, 4 equivalent). The suspension is stirred at 60° C. for 2 h 15 min. The reaction mixture is dilluted with 2 l ethyl acetate and 500 ml water is added. The pH is adjusted to 8-9 by addition of sodium bicarbonate. The layers are separated and the aqueous layer is reextracted with 500 ml ethyl acetate. The organic layers are dried with sodium sulfate, filtered and evaporated to a volume of 300 ml. A crystalline precipitate is formed and removed by filtration (side product). The filtrate is evaporated to 100 ml whereupon the product crystallizes to give 2-(2-chloro-pyrimidin-4-ylamino)-benzenesulfonamide (97% purity by HPLC). The mother liquor of this cristallisation is further purified by column chromatography and crystallisation to give further 2-(2-chloro-pyrimidin-4-ylamino)-benzenesulfonamide.

(b) 2-[2-(1H-Indazol-6-ylamino)-pyrimidin-4-ylamino]-benzenesulfonamide: To a suspension of 7.25 g (25.46 mmol) 2-(2-Chloro-pyrimidin-4-ylamino)-benzenesulfonamide and 4.07 g (30.55 mmol, 1.2 equivalent) 6-aminoindazole in 400 ml isopropanol is added 13 ml conc. HCI* (130 mmol, 5 equivalent). The suspension is refluxed for 4 h 30 min. The reaction mixture is dilluted with 1.5 l ethyl acetate and 1 l water is added. The pH is adjusted to 8-9 by addition of sodium bicarbonate. The layers are separated and the aqueous layer is re-extracted with 500 ml ethyl acetate. The organic layers are dried with sodium sulfate, filtered and evaporated to a volume of 300 ml. A crystalline precipitate (1.01 g) is formed and removed by filtration (side product). The filtrate is purified by chromatography on 200 g silica gel eluting with ethyl acetate/methanol 95/5 v/v. Upon evaporation crystalls are formed which are filtered to give the title compound.

¹H NMR (400 MHz, DMSO-d₆): □ 9.42 (s, 1 H), 8.34 (d, 1h), 8.28 (d, 1H), 8.27 (s, 1H), 7.93 (s, 1H, 7.88 (d, 1H), 7.62 (m, 2H), 7.32 (d, 1 H), 7.24 (t, 1H), 6.40 (d, 1H). MS mz (%): 382 (M+H, 100);

### EXAMPLE 2

### 2-[2-(3,4,5-Trimethoxy-phenylamino)-pyrimidin-4-ylamino]-benzenesulfonamide

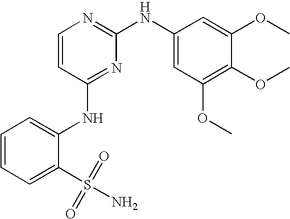

The title compound is prepared from 2-(2-chloro-pyrimidin-4-ylamino)-benzenesulfonamide as described in Example 1 using 3,4,5-Trimethoxy-phenylamine instead of 6-aminoindazole in step (b).

¹H NMR (400 MHz, DMSO-d₆): □ 9.18 (s, 1H), 8.22 (d, 1H), 8.17 (d, 1H), 7.89 (d, 1H), 7.55 (t, 1H), 7.25 (t, 1H), 7.14 (s, 2H), 6.40 (d, 1H), 3.69 (s, 6H), 3.62 (s, 3H). MS m/z (%): 432 (M+H, 100);

### EXAMPLE 3

### 2-methyl-6-[2-(3,4,5-Trimethoxy-phenylamino)-pyrimidin-4-ylamino]-benzenesulfonamide

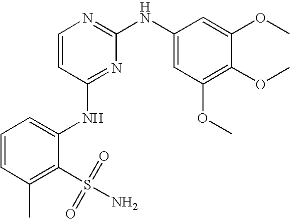

The tilte compound is prepared as described in Example 1 with the difference that in step (a) 2-amino-6-methyl-benzenesulfonamide is used instead of 2-aminobenzenesulfonamide. 2-Amino-6methyl-benzenesulfonamide may be prepared as described by Girard, Y el al.; J. J. Chem. Soc. Perkin Trans. I. 1979, 4, 1043-1047: Under an atmosphere of nitrogen m-toluidin (32.1 g, 32.5 ml, 0.30 mmol) is added dropwise to a solution of chlorosulfonyl isocyanate (51.3 ml, 83.6 g, 0.59 mmol) in nitroethane (400 ml) at −55−49° C. The cold bath is removed and the mixture allowed to warm to −8° C., whereupon aluminium chloride (51 g, 0.38 mmol) is added. Heating the mixture to 100° C. for 20 min forms a clear brown solution, which is cooled to RT and poured on ice. After filtration, washing with ice water and diethyl ether the precipitate is collected and dissolved in dioxane (300 ml). Water (1000 ml) and conc. HCI (1500 ml) are added to form a suspension, which is heated to 120° C. for 18 h. After cooling to RT the clear brown solution is washed with diethyl ether/hexane (1400 ml, 1/1 v/v) and adjusted to pH=8 by addition of sodium carbonate. Extraction using ethyl acetate (2×1000 ml), washing of the organic phase with water (500 ml) and brine (500 ml), drying (magnesium sulfate) and concentration yields a brown solid, which is purified by chromatography on silica using methylene chloride/ethanol (100/1 v/v) to yield the desired product as a white solid.

Melting point: 72-75° C. (Propan-2-ol); ¹H NMR (400 MHz, DMSO-d₆): □ 0 2.64 (s, 3H, Me), 3.63 (s, 3H, OMe), 3.68 (s, 6H, OMe), 6.31 (d, J=5 Hz, 1H, pyrimidine CH), 7.07 (d, J=8 Hz, 1H, arom. CH), 7.15 (s, 2H, arom. CH), 7.40 (t, J=8 Hz, 1H, arom. CH), 7.65 (s, 2H, SO₂NH₂), 8.04 (d, J=8 Hz, 1H, arom. CH), 8.12 (d, J=5 Hz, 1H, pyrimidine CH), 9.14 (s, 1H, NH), 9.40 (s, 1H, NH). MS (ES⁺) m/z: 446 (MH⁺), 468 (MNa⁺) MS (ES⁻): 444 (M-H)³¹

### EXAMPLE 4

### 2-Methoxy-6-[2-(3,4,5-trimethoxy-phenylamino)-pyrimidin-4-ylamino]-benzenesulfonamide

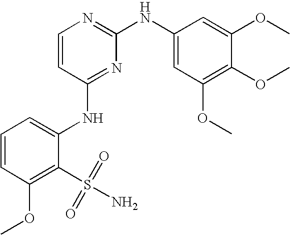

The title compound is prepared as described in Example 1 with the difference that in step (a) 2-amino-6-methoxy-benzenesulfonamide is used instead of 2-Amino-6-methyl-benzenesulfonamide. 2-Amino-6-methoxy-benzenesulfonamide may be prepared from 12.3 g of meta-anisidine following an analogous procedure as described in Example 1a. NMR (400 MHz, DMSO-d₆): □3.62 (s, 3H, OMe), 3.69 (s, 6H, OMe), 3.91 (s, 3H, OMe), 6.31 (d, J=5 Hz, 1H, pyrimidine CH), 6.86 (d, J=8 Hz, 1H, arom. CH), 7.12 (s, 2H, arom. CH), 7.43 (t, J=8 Hz, 1H, arom. CH), 8.01 (d, J=8 Hz, 1H, arom. CH), 8.11 (d, J=5 Hz, 1H, pyrimidine CH), 9.18 (s, 1H, NH), 9.79 (br, 1H, NH). MS (ES⁺): 462.2 (MH⁺), 484.2 (MNa⁺) MS (ES⁻): 460.3 (M-H)⁻

The compounds of formula X₁

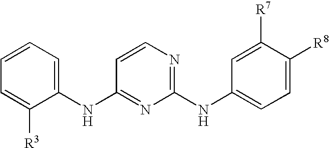

wherein R³, R⁷ and R⁸ are as defined in Table 1, may be prepared by following the procedure of Example 1 but using the appropriate starting materials.

TABLE 1

| | | | | MS Data | | |
|---|---|---|---|---|---|---|
| Example | R³ | R⁷ | R⁸ | *ES+ | *ES− | *EI |
| 5 | —OH | —O-(1-methyl)-azacyclohept- | —H | 406 | 404 | |
| | | 4-yl | | | | |
| 6 | —SO₂NH₂ | —O-(1-methyl)-azacyclohept- | —H | 469.3 | | |
| | | 4-yl | | | | |
| 7 | —SO₂NH₂ | —O-2-(1-methyl-azacyclopent- | —H | 469.3 | | |
| | | 2-yl)-ethyl | | | | |
| 8 | —OH | —O-2-(1-piperidyl)-ethyl | —OCH₃ | 436.3 | 434.4 | |
| 9 | —OH | —O-2-(1-methyl-azacyclopent- | —H | 406 | 404 | |
| | | 2-yl)-ethyl | | | | |
| 10 | —SO₂NH₂ | —O—CH₂CH₂CH₂-1-imidazolyl | —OCH₃ | 496 | 494 | |
| 11 | —SO₂NH₂ | —O-2-(1-piperidyl)-ethyl | —OCH₃ | 499.2 | 497.3 | |
| 12 | —SO₂NH₂ | —O—CH₂CH₂-1-methyl- | —H | 466 | 464 | |
| | | imidazol-1-yl | | | | |
| 13 | —OH | —O-2-[1-(1,2,4-triazolyl)]-ethyl | —H | 390 | 388 | |
| 14 | —OH | —O-2-hydroxyethyl | —OCH₃ | 369.4 | 367.3 | |
| 15 | —SO₂NH₂ | —O-2-hydroxyethyl | —OCH₃ | | | 431 |
| 16 | —SO₂NH₂ | —O—CH₂CH₂-1-imidazolyl | —OCH₃ | | | |
| 17 | —SO₂NH₂ | —O-2-[1-(1,2,4-triazolyl)]-ethyl | —H | | | 452 |
| 18 | —SO₂NH₂ | —NH—N═N— | | | 381 | |
| 19 | —SO₂NHCH₃ | —O—CH₂CH₂-1-imidazolyl | —OCH₃ | 496 | 494 | |
| 20 | —SO₂NH₂ | —O-2-(1-piperidyl)-ethyl | —H | 469 | 467 | |
| 21 | —SO₂NH₂ | —O—CH₂CH₂-1-imidazolyl | —H | 452 | 450 | |
| 22 | —OH | —O-2-(1-piperidyl)-ethyl | —H | 406 | | |
| 23 | —COOH | -4-morpholino | —H | | | |
| 24 | —OH | —O—CH₂CH₂CH₂-1-imidazolyl | —OCH₃ | 433 | 431 | |
| 25 | —SO₂NHCH₃ | —CH═N—NH— | | 396 | 394 | |
| 26 | —SO₂NH₂ | —O-2-(4-morpholino)ethyl | —H | 471 | 469 | |
| 27 | —SO₂NH₂ | —OCH₃ | —OCH₃ | 402 | 400 | |
| 28 | —OH | —O-2-(4-morpholino)ethyl | —H | 408 | 406 | |
| 29 | —SO₂NH₂ | —CH═N—NH— | | | | 381 |
| 30 | —SO₂NHCH₃ | —O—CH₂CH₂-1-imidazolyl | —H | | | |
| 31 | —COOH | Amino | —H | 322 | | |
| 32 | —SO₂NH₂ | —O—CH₂CH₂CH₂-1-imidazolyl | —H | 466.2 | 464.3 | |
| 33 | —COOH | —N(CH₃)₂ | —H | | | |
| 34 | -5-(1,2,3,4- | —NH—C(O)CH₃ | —H | 388 | 386 | |
| | tetrazolyl) | | | | | |
| 35 | —SO₂NHCH₃ | —NH—N═CH— | | | | |
| 36 | —COOH | —OH | —H | | | |
| 37 | —COOH | —H | -4- | | | |
| | | | piperidyl | | | |
| 38 | —COOH | —CH₂—OH | —H | | | |
| 39 | —OH | —O—CH₂CH₂-1-imidazolyl | —OCH3 | | | |
| 40 | —SO₂NH—CH₂CH₂—OH | —O—CH₂CH₂-1-imidazolyl | —H | 496 | 494 | |
| 41 | —C(O)NH₂ | Amino | —H | 321 | | |
| 42 | —SO₂NH₂ | —CH═CH—NH— | | 381 | | |
| 43 | -5-(1,2,3,4- | —NHCH₂-3-pyridyl | —H | | 435 | |
| | tetrazolyl) | | | | | |
| 44 | —SO₂NH₂ | —NH—CH═CH— | | | 379 | |
| 45 | —COOH | —H | -4- | | | |
| | | | morpholino | | | |
| 46 | —COOH | —H | -1-(4- | | | |
| | | | amino)- | | | |
| | | | piperidyl | | | |
| 47 | —SO₂NH₂ | —OCH₃ | —H | 372 | 370 | |
| 48 | —SO₂N(CH₃)₂ | —O—CH₂CH₂-1-imidazolyl | —H | 480 | 478 | |

The compounds of formula X₂

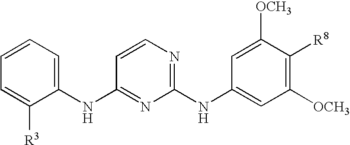

wherein R³ and R⁸ are as defined in Table 2, may be prepared by following the procedure of Example 1 but using the appropriate starting materials.

TABLE 2

| | | | MS Data | |
|---|---|---|---|---|
| Example | R³ | R⁸ | *ES+ | *ES− |
| 49 | —COOH | —OCH₃ | 397 | 395 |
| 50 | —SO₂NH₂ | —OH | | |
| 51 | —SO₂NHCH₃ | —OCH₃ | | |
| 52 | -5-(1,2,3,4-tetrazolyl) | —OCH₃ | 421 | |
| 53 | —SO₂NH-cyclopropyl | —OCH₃ | 472.2 | 470.3 |
| 54 | —C(O)NHOH | —OCH₃ | 412 | 410 |
| 55 | —SO₂NH—CH₂CH₂—OH | —OCH₃ | 476 | 474 |
| 56 | —SO₂N(CH₃)₂ | —OCH₃ | 460.3 | 458.3 |
| 57 | —OH | —OCH₃ | 369 | 367 |
| 58 | —SO₂NH—CH₂CH₂CH₃ | —OCH₃ | 474 | 472 |
| 59 | —CH₂OH | —OCH₃ | | |
| 60 | —SO₂NH₂ | —H | 402 | |

The compounds of formula X₃

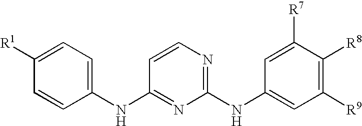

wherein R¹, R⁷, R³ and R⁹ are as defined in Table 3, may be prepared by following the procedure of Example 1 but using the appropriate starting materials.

TABLE 3

| | | | | | MS Data | |
|---|---|---|---|---|---|---|
| Example | R¹ | R⁷ | R⁸ | R⁹ | *ES+ | *ES− |
| 61 | —SO₂NH—CH₂CH₂—O—CH₂CH₂—OH | —H | —N(CH₃)—C(O)CH₃ | —H | | |
| 62 | —SO₂NH₂ | —OCH₃ | —OCH₃ | —OCH₃ | | |
| 63 | —SO₂NH₂ | —O—CH₂CH₂-1- | —OCH₃ | —H | | |
| | | imidazolyl | | | | |
| 64 | —SO₂NH—CH₂CH₂—O—CH₂CH₂—OH | —OCH₃ | —OCH₃ | —OCH₃ | 520 | 518 |
| 65 | —N(CH₃) C(O)CH₃ | —OCH₃ | —OCH₃ | —OCH₃ | 424 | 422 |
| 66 | —CH₂CH₂—OH | —SO₂NH—CH₂CH₂CH₂CH₃ | —H | —H | | |
| 67 | —SO₂NH₂ | —OCH₃ | —H | —OCH₃ | | |
| 68 | —SO₂NH₂ | —O—CH₂CH₂-1- | —H | —H | | |
| | | imidazolyl | | | | |
| 69 | —CH₂CH₂—OH | —O—CH₂CH₂-1- | —H | —H | | |
| | | imidazolyl | | | | |
| 70 | —CH₂CH₂—OH | —OCH₃ | —H | —OCH₃ | | |
| 71 | —SO₂NH₂ | —OH | —H | —H | | |
| 72 | —O—CH₂CH₂—OH | —O—CH₂CH₂-1- | —H | —H | | |
| | | imidazolyl | | | | |
| 73 | —SO₂NH-2-thiazolyl | —OCH₃ | —OCH₃ | —OCH₃ | 515 | 513 |

The compounds of formula X₄

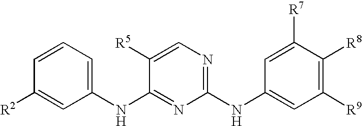

wherein R², R⁵, R⁷, R⁸ and R⁹ are as defined in Table 4, may be prepared by following the procedure of Example 1 but using the appropriate starting materials.

TABLE 4

| | | | | | | MS Data | |
|---|---|---|---|---|---|---|---|
| Example | R² | R⁵ | R⁷ | R⁸ | R⁹ | *ES+ | *ES− |
| 74 | —SO₂NH-2- | —H | —OCH₃ | —OCH₃ | —OCH₃ | 472 | 470 |
| | propenyl | | | | | | |
| 75 | —SO₂NH₂ | —H | —OCH₃ | —OCH₃ | —OCH₃ | | |
| 76 | —OH | —H | —O-(1-methyl)- | —H | —H | 406.3 | 404.3 |
| | | | azacyclohept-4-yl | | | | |
| 77 | —OH | —H | —O—CH₂CH₂—OH | —OCH₃ | —H | 369 | 367 |
| 78 | —SO₂NH₂ | —Br | —OCH₃ | —OCH₃ | —OCH₃ | 510.1/ | 508.1/ |
| | | | | | | 512.1 | 510.2 |
| 79 | —SO₂NH₂ | —H | —CH═N—NH— | | —H | 382 | |
| 80 | —SO₂NH₂ | —CH₃ | —OCH₃ | —OCH₃ | —OCH₃ | 446 | 444 |
| 81 | —SO₂NH₂ | —H | —O—CH₂CH₂-1- | —OCH₃ | —H | 482 | 480 |
| | | | imidazolyl | | | | |
| 82 | —OH | —H | —O—CH₂CH₂-1-piperidyl | —OCH₃ | —H | 436.3 | 434.3 |
| 83 | —OH | —H | —O—CH₂CH₂-1- | —OCH₃ | —H | 419 | 417 |
| | | | imidazolyl | | | | |
| 84 | —SO₂NH₂ | —H | —O—CH₂CH₂-1- | —H | —H | 452 | 450 |
| | | | imidazolyl | | | | |
| 85 | —CH₃ | —C≡N | —OCH₃ | —OCH₃ | —OCH₃ | 392 | |
| 86 | —SO₂NH₂ | —H | —NH—N═CH— | | —H | 382 | |
| 87 | —OH | —H | —OCH₃ | —OCH₃ | —OCH₃ | 369 | 367 |
| 88 | —SO₂NHCH₃ | —CH₃ | —OCH₃ | —OCH₃ | —OCH₃ | 460 | 458 |
| 89 | —OH | —H | —OH | COOH | —OCH₃ | | |
| 90 | —OH | —H | —O—CH₂CH₂-1-piperidyl | —H | —H | 406 | 404 |
| 91 | —SO₂NH-2- | —H | —O—CH₂CH₂-1- | —H | —H | 492.3 | 490.3 |
| | propenyl | | imidazolyl | | | | |
| 92 | —SO₂NH₂ | —Br | —O—CH₂CH₂-1-(1- | —H | —H | 544.1/ | 542/ |
| | | | methyl)-imidazolyl | | | 546 | 544.2 |
| 93 | —SO₂NH₂ | —H | —O—CH₂CH₂—OH | —OCH₃ | —H | | |
| 94 | —OH | —H | —O-(1-methyl)- | —H | —H | | |
| | | | azacyclopent-2-yl | | | | |
| 95 | —OH | —H | —O—CH₂CH₂-1- | —H | —H | 389 | 387 |
| | | | imidazolyl | | | | |
| 96 | —OH | —H | —O—CH₂CH₂CH₂-1- | —OCH₃ | —H | 433.4 | 431.4 |
| | | | imidazolyl | | | | |
| 97 | —SO₂NH₂ | —H | —OCH₃ | —H | —OCH₃ | | |
| 98 | —OH | —H | —OCH₃ | —OCH₃ | —H | 339 | 337 |
| 99 | —SO₂NHCH₂—CH₂CH₂CH₃ | —H | —OCH₃ | —OCH₃ | —OCH₃ | 488 | 486 |
| 100 | —SO₂NH—CH₃ | —CH₃ | —O—CH₂CH₂-1- | —OCH₃ | —H | 510 | 508 |
| | | | imidazolyl | | | | |
| 101 | —SO₂NHCH₂—CH₂CH₂CH₃ | —H | —O—CH₂CH₂-1- | —H | —H | 08 | 506 |
| | | | imidazolyl | | | | |
| 102 | —OH | —H | —O—CH₂CH₂-4- | —H | —H | 408 | |
| | | | morpholino | | | | |
| 103 | —OH | —H | —NH—N═CH— | | —H | 319 | 317 |
| 104 | —OH | —H | —CHN—NH— | | —H | 319 | 317 |
| 105 | —OH | —H | —O—CH₂CH₂-1- | —H | —H | | |
| | | | imidazolyl | | | | |
| 106 | —SO₂NH—CH₃ | —CH₂—CH₃ | —OCH₃ | —OCH₃ | —OCH₃ | 474.3 | 472.3 |
| 107 | —SO₂NH₂ | —H | —OCH₃ | —OCH₃ | —OCH₃ | | |

The compounds of formula X₅

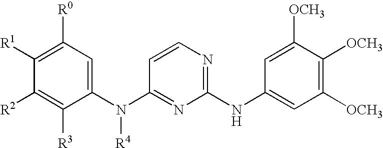

wherein R¹⁰, R¹, R², R³ and R⁴ are as defined in Table 5, may be prepared by following the procedure of Example 1 but using the appropriate starting materials.

TABLE 5

| | | | | | | MS Data | |
|---|---|---|---|---|---|---|---|
| Example | R⁰ | R¹ | R² | R³ | R⁴ | *ES+ | *ES− |
| 108 | —H | —OCH₃ | —OH | —H | —H | | |
| 109 | —H | nitro | —H | —OH | —H | 414 | 412 |
| 110 | —H | —N═CH—CH═CH— | | —H | —H | | |
| 111 | —H | —CH═N—NH— | | —H | —H | 393 | 391 |
| 112 | —H | —NH—N═CH— | | —H | —H | 393 | |
| 113 | —H | —H | —OH | —CH₂CH₂CH₂— | | 409 | 407 |
| 114 | —CH₃ | —H | —CH₃ | —OH | —H | 397 | |
| 115 | —H | phenyl | —H | —SO₂NH₂ | —H | 508 | 506 |
| 116 | —CH₃ | —H | —H | —SO₂NH₂ | —H | 446 | 444 |

The compounds of formula X₆

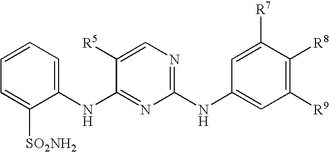

wherein R⁵, R⁷, R⁸ and R⁹ are as defined in Table 6, may be prepared by following the procedure of Example 1 but using the appropriate starting materials.

TABLE 6

| Example | R⁵ | R⁷ | R⁸ | R⁹ | *ES+ | *ES− |
|---|---|---|---|---|---|---|
| 117 | —CH₃ | —O—CH₂CH₂-1-imidazolyl | —H | —H | 466 | |
| 118 | —CH₂CH₃ | —OCH₃ | —OCH₃ | —OCH₃ | 460 | 458 |
| 119 | —Br | —NH—N═CH— | | —H | 461 | |
| 120 | —CH₃ | —O—CH₂CH₂-1-imidazolyl | —OCH₃ | —H | 496 | |
| 121 | —CH₃ | —OCH₃ | —OCH₃ | —OCH₃ | 446 | |
| 122 | —CH₃ | —N═N—NH— | | —H | 397.2 | 395.2 |
| 123 | —CH₃ | —O—CH₂CH₂-1-methyl-imidazol- | —H | —H | 480 | |
| | | 1-yl | | | | |
| 124 | —Br | —CH═N—NH— | | —H | 461.3 | 458.1/ |
| | | | | | | 460 |
| 125 | —CH₃ | —NH—N═CH— | | —H | 396 | |
| 126 | —Br | —OCH₂CH₂-(4-methyl-piperazin- | —H | —H | 562/ | 560/ |
| | | 1-yl) | | | 564 | 562 |

The compounds of formula X₇

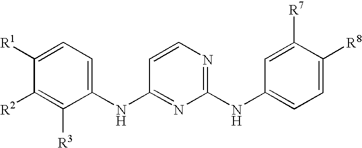

wherein R¹, R², R³, R⁷ and R⁸ are as defined in Table 7, may be prepared by following the procedure of Example 1 but using the appropriate starting materials.

TABLE 7

| Ex | R¹ | R² | R³ | R⁷ | R⁸ | *ES+ | *ES− |
|---|---|---|---|---|---|---|---|
| 127 | —OCH₃ | —OH | —H | —OH | —OCH₃ | | |
| 128 | —H | —CH₃ | —SO₂NH₂ | —O—CH₂CH₂-1-imidazolyl | —H | 466 | 464 |
| 129 | —OCH₃ | —OH | —H | —O—CH₂CH₂-1-imidazolyl | —OCH₃ | | |
| 130 | —OCH₃ | —OH | —H | —O—CH₂CH₂—OH | —OCH₃ | 399 | 397 |
| 131 | —OCH₃ | —OH | —H | —O—(1-methyl-azacyclohept-4- | —H | 436 | |
| | | | | yl) | | | |
| 132 | —CH₃ | —H | —SO₂NH₂ | —O—CH₂CH₂-1-imidazolyl | —H | 466 | 464 |
| 133 | —OCH₃ | —OH | —H | —O—CH₂CH₂-(1-methyl)- | —H | 436 | 434 |
| | | | | azacyclopent-2-yl | | | |
| 134 | —OCH₃ | —OH | —H | —CF₃ | —H | | |
| 135 | —N═CH—CH═CH— | | —H | —O—CH₂CH₂-1-imidazolyl | —OCH₃ | | |
| 136 | —OCH₃ | —OH | —H | —O—CH₂CH₂CH₂-1-imidazolyl | —OCH₃ | 463 | 461 |
| 137 | —OCH₃ | —OH | —H | —O—CH₂CH₂-1-piperidyl | —OCH₃ | 466.4 | 464.4 |
| 138 | —CH═N—NH— | | —H | —NH—N═CH— | | | |
| 139 | —CH═N—NH— | | —H | —CH—N═NH— | | | |
| 140 | —OCH₃ | —OH | —H | —O—CH₂CH₂-1-piperidyl | —H | 436 | 434 |
| 141 | —H | —OCH₃ | —SO₂NH₂ | —O—CH₂CH₂-1-pyrrolidinyl | —H | 485.3 | 483.3 |
| 142 | —H | —OCH₃ | —SO₂NH₂ | —O—CH₂CH₂-1-pyrrolidinyl | —CH₃ | 499.2 | 497.3 |
| 143 | —H | —OCH₃ | —SO₂NH₂ | —O—CH₂CH₂CH₂-morpholino | —OCH₃ | 545.2 | 545.3 |
| 144 | —H | —OCH(CH₃)₂ | —SO₂NH₂ | —O—CH₂CH₂—(4-methyl- | —OCH₃ | 572.2 | 570.3 |
| | | | | piperazin-1-yl | | | |
| 145 | —H | —OCH₃ | —SO₂NH₂ | —O—CH₂CH₂-1-piperidinyl | —H | 499.2 | 497.3 |
| 146 | —CH₃ | —OCH₃ | —SO₂NH₂ | —O—CH₂CH₂CH₂-1-pyrrolidinyl | —OCH₃ | 543.2 | |
| 147 | —CH₃ | —OCH₃ | —SO₂NH₂ | —O—CH₂CH₂CH₂-1-pyrrolidinyl | —H | 513.2 | 511.2 |
| 148 | —H | —OCH(CH₃)₂ | —SO₂NH₂ | —O—CH₂CH₂-1-piperidinyl | —H | 527.2 | 525.3 |
| 149 | —H | —CH₃ | —SO₂NH₂ | —N(CH₃)₂ | —OCH₃ | 429.3 | 427.3 |
| 150 | —OCH₃ | —CH₃ | —SO₂NH₂ | —O—CH₂CH₂CH₂-1-pyrrolidinyl | —OCH₃ | 527.2 | 525.3 |
| 151 | —OCH₃ | —H | —SO₂NH₂ | —O—CH₂CH₂CH₂-1-pyrrolidinyl | —OCH₃ | 529.2 | 527.3 |
| 152 | —H | —F | —SO₂NH₂ | —N(CH₃)₂ | —OCH₃ | 433.1 | |
| 153 | —H | —CH₃ | —SO₂NH₂ | —O—CH₂CH₂-(1-methyl- | —H | | |
| | | | | pyrrolidin-2-yl) | | | |
| 154 | —H | —OCH₃ | —SO₂NH₂ | —O—CH₂CH₂—OH | —H | 432.2 | 430.2 |
| 155 | —H | —CH₃ | —SO₂NH₂ | —O—CH₂CH₂-(1-methyl- | —OCH₃ | 513.2 | 511.3 |
| | | | | pyrrolidin-2-yl) | | | |
| 156 | —OCH₃ | —H | —SO₂NH₂ | —O—CH₂CH₂-1-piperidinyl | —H | 499.2 | 497.3 |
| 157 | —OCH₃ | —H | —SO₂NH₂ | —O—CH₂CH₂-1-pyrrolidinyl | —OCH₃ | 515.2 | 513.2 |
| 158 | —H | —CH₃ | —SO₂NH₂ | —O—CH₂CH₂—OH | —OCH₃ | 446.2 | 444.2 |
| 159 | —OC₂H₅ | —H | —SO₂NH₂ | —O—CH₂CH₂-1-pyrrolidinyl | —CH₃ | 513.3 | 511.3 |
| 160 | —OCH₃ | —OCH₃ | —SO₂NH₂ | —O—CH₂CH₂-(4-methyl- | —OCH₃ | 574.2 | 572.2 |
| | | | | piperazin-1-yl) | | | |
| 161 | —H | —Cl | —SO₂NH₂ | -(4-methyl-piperazin-1-yl) | —H | 474.5 | 472.5 |
| 162 | —H | —CH₃ | —SO₂NH₂ | —O—CH₂CH₂-(4-cyclopentyl- | —H | 552.3 | 550.3 |
| | | | | piperazin-1-yl) | | | |
| 163 | —CH═CH—CH═CH— | | —SO₂NH₂ | -(4-methyl-piperazin-1-yl) | —H | 490.5 | 488.4 |
| 164 | —H | —H | —SO₂NH₂ | —O—CH₂CH₂-piperazin-1-yl | —H | 470.2 | 468.3 |
| 165 | —H | —OCH₃ | —SO₂NH₂ | —H | —OCH₃ | 402.2 | 400.2 |
| 166 | —H | —OCH₃ | —SO₂NH₂ | —O—CH₂CH₂-(4-benzyl- | —H | 590.3 | 588.3 |
| | | | | piperazin-1-yl) | | | |
| 167 | —CH₃ | —H | —SO₂NH₂ | —O—CH₂CH₂-1-pyrrolidinyl | —H | 469.2 | 467.3 |
| 168 | —Br | —H | —SO₂NH₂ | —O—CH₂CH₂-1-piperidinyl | —H | 549.1 | 547.2 |

The compounds of formula X₈

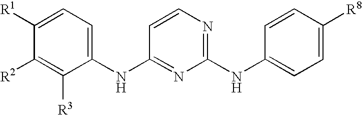

wherein R¹, R², R³ and R⁸ are as defined in Table 8, may be prepared by following the procedure of Example 1 but using the appropriate starting materials.

TABLE 8

| Ex | R¹ | R² | R³ | R⁸ | *ES+ | *ES− |
|---|---|---|---|---|---|---|
| 169 | 4-morpholino | —H | —H | —H | | |
| 170 | —CH═N—NH— | | —H | —H | 363 | 361 |
| 171 | —OCH₃ | —OH | —H | —H | | |
| 172 | —CH₃ | —H | —SO₂NH₂ | —OCH₃ | 446 | |

The compounds of formula X₉

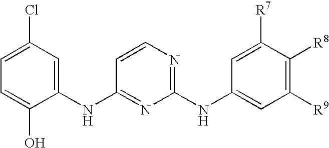

wherein R⁷, R⁸ and R⁹ are as defined in Table 9, may be prepared by following the procedure of Example 1 but using the appropriate starting materials.

TABLE 9

| Example | R⁷ | R⁸ | R⁹ | *ES+ | *ES− |
|---|---|---|---|---|---|
| 173 | —O—CH₂CH₂-1-piperidyl | —OCH₃ | —H | 470.3 | 468.3 |
| 174 | —O—(1-methyl-azacyclohept-4- | —H | —H | 440 | |
| | yl) | | | | |
| 175 | —O—(1-methyl-azacyclopent-2- | —H | —H | 440 | 438 |
| | yl) | | | | |
| 176 | —O—CH₂CH₂—CH₂-1-imidazolyl | —OCH₃ | —H | 467 | 465 |
| 177 | —OCH₃ | —OCH₃ | —OCH₃ | | |
| 178 | —O—CH₂CH₂-1-(1,2,4-triazolyl) | —H | —H | 424 | 422 |
| 179 | —O—CH₂CH₂-1-piperidyl | —H | —H | | |
| 180 | —O—CH₂CH₂—OH | —OCH₃ | —H | | |
| 181 | —O—CH₂CH₂-4-morpholino | —H | —H | 442 | 440 |
| 182 | —O—CH₂CH₂CH₂-1-imidazolyl | —H | —H | | |

The compounds of formula X,₁₀

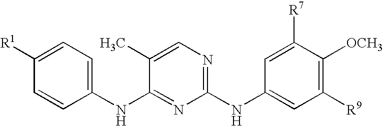

wherein R¹, R⁷ and R⁹ are as defined in Table 10, may be prepared by following the procedure of Example 1 but using the appropriate starting materials.

TABLE 10

| EX | R¹ | R⁷ | R⁹ | *ES+ | *ES− |
|---|---|---|---|---|---|
| 183 | —CH₂CH₂—OH | —OCH₃ | —OCH₃ | 411 | 409 |
| 184 | —SO₂NH₂ | —O—CH₂CH₂-1- | —H | 496.3 | 494.3 |
| | | imidazolyl | | | |

The compounds of formula X₁₁ 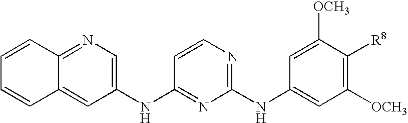
wherein R⁸ is —OCH₃ (Example 185) or —OH (Example 186), may be prepared by following the procedure of Example 1 but using the appropriate starting materials.

The compounds of formula X₁₂

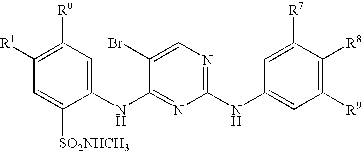

wherein R⁰, R¹, R⁷, R⁸ and R⁹ are as defined in Table 12, may be prepared by following the procedure of Example 1 but using the appropriate starting materials.

TABLE 12

| Example | R⁰ | R¹ | R⁷ | R⁸ | R⁹ |
|---|---|---|---|---|---|
| 187 | —H | —H | —H | —SO₂NH₂ | —H |
| 188 | —H | —H | —H | —H | —CH₃ |
| 189 | —H | —H | —H | —CH₃ | —H |
| 190 | —H | —F | —OCH₃ | —OCH₃ | —OCH₃ |
| 191 | —H | —H | —H | —CH₃ | —CH₃ |
| 192 | —H | —H | —CH₃ | —H | —CH₃ |
| 193 | —H | —H | —OCH₃ | —CH₃ | —H |
| 194 | —H | —H | —H | —H | —N(CH₃)₂ |
| 195 | —H | —H | —OCH(CH₃)₂ | —H | —H |
| 196 | —H | —H | —H | —OCH(CH₃)₂ | —H |
| 197 | —H | —H | —CH(CH₃)₂ | —H | —H |
| 198 | —H | —H | —H | —CH═N—NH— | |
| 199 | —H | —H | —OCH₃ | —CH₃ | —OCH₃ |
| 200 | —OCH₃ | —H | —OCH₃ | —OCH₃ | —OCH₃ |
| 201 | —H | —H | —H | —H | —H |
| 202 | —CH₃ | —Cl | —OCH₃ | —OCH₃ | —OCH₃ |
| 203 | —H | —H | —H | —H | —CF₃ |
| 204 | —Cl | —CH₃ | —OCH₃ | —OCH₃ | —OCH₃ |

| | | | | |
|---|---|---|---|---|
| 205 | —H | —H | —H | —NH—CH═N— |
| 206 | —H | —H | —H | —N(—CH₂CH₂CH₂-4-morpholino)—CH═CH— |

| | | | | |
|---|---|---|---|---|
| 207 | —H | —H | —CH₂CH₂—CH₂— | —H |

The compounds of formula X₁₃

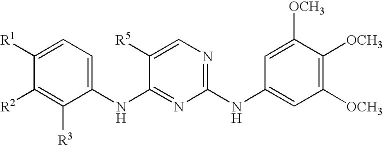

wherein R¹, R², R³ and R5 are as defined in Table 13, may be prepared by following the procedure of Example 1 but using the appropriate starting materials.

TABLE 13

| Example | R¹ | R² | R³ | R⁵ | *ES+ | *ES− |
|---|---|---|---|---|---|---|
| 208 | —H | —H | —SO₂NHCH₃ | —CF₃ | 514.0 | |
| 209 | —H | —H | —SO₂NHC₃H₇ | —Br | | |
| 210 | —H | —H | —SO₂NH—CH₂CH-cyclopropyl | —Br | | |
| 211 | —H | —H | —SO₂NHCH₃ | —CH₃ | | |
| 212 | —H | —H | —SO₂N(CH₃)₂ | —Br | | |
| 213 | —H | —H | —SO₂NHCH₃ | —Cl | | |
| 214 | —H | —H | —SO₂NHCH₃ | —I | | |
| 215 | —H | —H | —SO₂NHCH₃ | —Br | | |
| 216 | —CH₃ | —OCH₃ | —SO₂NH₂ | —H | 476 | 474 |
| 217 | —H | piperidino | —SO₂NH₂ | —H | 515.5 | 513.4 |
| 218 | —H | morpholino | —SO₂NH₂ | —H | 517.4 | 515.4 |
| 219 | —H | —C₂H₅ | —SO₂NH₂ | —H | | |
| 220 | —H | —CH₃ | —SO₂NH₂ | —Cl | | |
| 221 | —H | —CH₃ | —SO₂NHCH₃ | —H | 460.4 | |
| 222 | —H | phenyl | —SO₂NH₂ | —H | 508.2 | 506.3 |

The compounds of formula X₁₄

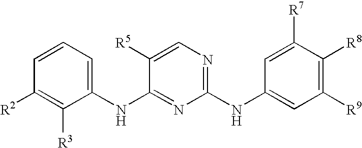

wherein R², R³, R⁵, R⁷, R⁸ and R⁹ are as defined in Table 14, may be prepared by following the procedure of Example 1 but using the appropriate starting materials.

TABLE 14

| Ex | R² | R³ | R⁵ | R⁷ | R⁸ | R⁹ | *ES+ | *ES− |
|---|---|---|---|---|---|---|---|---|
| 223 | —OCH₃ | —SO₂NH₂ | —H | —H | —CH═N—N(CH₃)— | | | 424 |
| 224 | —OCH₃ | —SO₂NH₂ | —H | —O—CH₂CH₂—OCH₃ | —OCH₃ | —H | 476.2 | 474.3 |
| 225 | —OCH(CH₃)₂ | —SO₂NH₂ | —H | —O—CH₂CH₂— | —OCH₃ | —H | 551.2 | 555.3 |
| | | | | piperidino | | | | |
| 226 | —OCH₃ | —SO₂NH₂ | —H | —O—CH₂CH₂—(4- | —H | —H | 514.3 | 512.3 |
| | | | | methyl-piperazin-1- | | | | |
| | | | | yl) | | | | |
| 227 | —OCH₃ | —SO₂NH₂ | —H | -morpholino | —OCH₃ | —H | 487.1 | 485.2 |
| 228 | —CH₃ | —SO₂NH₂ | —H | —O—CH₂CH₂CH₂- | —OCH₃ | —H | 527.3 | |
| | | | | piperidino | | | | |
| 229 | —CH₃ | —SO₂NH₂ | —H | —O—CH₂CH₂CH₂-1- | —OCH₃ | —H | 513.2 | 511.3 |
| | | | | pyrrolidinyl | | | | |
| 230 | —O—CH₂CH₂—OCH₃ | —SO₂NH₂ | —H | —H | —CH═N—N(CH₃)— | | 539 | 537 |
| 231 | -(4-methyl- | —SO₂NH₂ | —H | —OCH₃ | —OCH₃ | —OCH₃ | 530.4 | 528.4 |
| | piperazin-1-yl) | | | | | | | |
| 232 | —OCH₃ | —SO₂NH₂ | —H | —O—CH₂CH₂—OH | —OCH₃ | —H | 462.2 | 460.3 |
| 233 | —OCH₃ | —SO₂NH₂ | —Br | —O—CH₂CH₂—OCH₃ | —OCH₃ | —H | | |
| 234 | —CH₃ | —SO₂NH₂ | —H | —O—CH₂CH₂-4- | —OCH₃ | —H | 528.2 | 526.3 |
| | | | | methyl-piperazin-1- | | | | |
| | | | | yl) | | | | |
| 235 | —CH₃ | —SO₂NH₂ | —H | —O—CH₂CH₂—N(CH₃)₂ | —H | —H | 443.2 | 441.3 |
| 236 | —H | —SO₂NH₂ | —H | —O—CH₂CH₂-1- | —OCH₃ | —H | 485.2 | 483.3 |
| | | | | pyrrolidinyl | | | | |
| 237 | —CH₃ | —SO₂NH₂ | —H | —H | —N(CH₃)—N═CH— | | 410 | |
| 238 | —CH₃ | —SO₂NH₂ | —H | —CH₃ | —OCH₃ | OCH₃ | | |
| 239 | —CH₃ | —SO₂NH₂ | —Br | —O—CH₂CH₂—OCH₃ | —OCH₃ | —H | 538/540 | |
| 240 | —OCH₃ | —SO₂NH₂ | —H | —OCH₃ | —H | —H | 402.2 | 400.2 |
| 241 | —H | —SO₂NH₂ | —H | —H | —CO—NH—CH₂CH₂—OCH₃ | —H | | |

ES+ means electrospray MS positive mode;
ES− means electrospray MS negative mode; and
EL means electron impact MS.

The compounds of formula l and their pharmaceutically acceptable salts, exhibit valuable pharmacological properties when tested in in vitro assays, and are therefore useful as pharmaceuticals. They are effective especially as protein tyrosine kinase inhibitors; they exhibit, for example, powerful inhibition of the tyrosine kinase activity of anaplastic lymphoma kinase (ALK) and the fusion protein of NPM-ALK . This protein tyrosine kinase results from a gene fusion of nucleophosmin (NPM) and the anaplastic lymphoma kinase (ALK), rendering the protein tyrosine kinase activity of ALK ligand-independent. NPM-ALK plays a key role in signal transmission in a number of hematopoetic and other human cells leading to hematological and neoplastic diseases, for example in anaplastic large-cell lymphoma (ALCL) and non-Hodgkin's lymphomas (NHL), specifically in ALK+NHL or Alkomas, in inflammatory myofibroblastic tumors (IMT) and neuroblastomas. (Duyster J et al. 2001 Oncogene 20, 56235637). In addition to NPM-ALK other gene fusions have been identified in human hematological and neoplastic diseases; mainly TPM3-ALK (a fusion of nonmuscle tropomyosin with ALK).

The ALK inhibitory activity and inhibitory activity against ALK-containing gene fusions of the compounds described herein make them useful pharmaceutical agents for the treatment of proliferative diseases. A proliferative disease is mainly a tumor disease (or cancer) (and/or any metastases). The inventive compounds are particularly useful for treating a tumor which is a breast cancer, genitourinary cancer, lung cancer, gastrointestinal cancer, epidermoid cancer, melanoma, ovarian cancer, pancreas cancer, neuroblastoma, head and/or neck cancer or bladder cancer, or in a broader sense renal, brain or gastric cancer; in particular (i) a breast tumor; an epidermoid tumor, such as an epidermoid head and/or neck tumor or a mouth tumor; a lung tumor, for example a small cell or non-small cell lung tumor; a gastrointestinal tumor, for example, a colorectal tumor; or a genitourinary tumor, for example, a prostate tumor (especially a hormone-refractory prostate tumor); or (ii) a proliferative disease that is refractory to the treatment with other chemotherapeutics; or (iii) a tumor that is refractory to treatment with other chemotherapeutics due to multidrug resistance.

In a broader sense of the invention, a proliferative disease may furthermore be a hyperproliferative condition such as leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. Proliferative diseases treated according to the present method include tumors of blood and lymphatic system (e.g. Hodgkin's disease, Non-Hodgkin's lymphoma, Burkitt's lymphoma, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoletic and related tissues, for example diffuse large cell lymphoma, T-cell lymphoma or cutaneous T-cell lymphoma). Myeloid cancer includes e.g. acute or chronic myeloid leukaemia.

Where a tumor, a tumor disease, a carcinoma or a cancer are mentioned, also metastasis in the original organ or tissue and/or in any other location are implied alternatively or in addition, whatever the location of the tumor and/or metastasis.

The compound is selectively toxic or more toxic to rapidly propiferating cells than to normal cells, particularly in human cancer cells, e.g., cancerous tumors, the compound has significant antiproliferative effects and promotes differentiation, e.g., cell cycle arrest and apoptosis.

The compounds of the present invention may be administered alone or in combination with other anticancer agents, such as compounds that inhibit tumor angiogenesis, for example, the protease inhibitors, epidermal growth factor receptor kinase inhibitors, vascular endothelial growth factor receptor kinase inhibitors and the like; cytotoxic drugs, such as antimetabolites, like purine and pyrimidine analog antimetabolites; antimitotic agents like microtubule stabilizing drugs and antimitotic alkaloids; platinum coordination complexes; anti-tumor antibiotics; alkylating agents, such as nitrogen mustards and nitrosoureas; endocrine agents, such as adrenocorticosteroids, androgens, anti-androgens, estrogens, anti-estrogens, aromatase inhibitors, gonadotropin-releasing hormone agonists and somatostatin analogues and compounds that target an enzyme or receptor that is overexpressed and/or otherwise involved a specific metabolic pathway that is upregulated in the tumor cell, for example ATP and GTP phosphodiesterase inhibitors, protein kinase inhibitors, such as serine, threonine and tyrosine kinase inhibitors, for example, Abelson protein tryosine kinase and the various growth factors, their receptors and kinase inhibitors therefore, such as, epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors and the like; methionine aminopeptidase inhibitors, proteasome inhibitors, and cyclooxygenase inhibitors, for example, cyclooxygenase-1 or -2 inhibitors. Such antiproliferative agents further include, aromatase inhibitors, antiestrogens, topoisomerase I inhibitors, topoisomerase II inhibitors, microtubule active agents, alkylating agents, histone deacetylase inhibitors, famesyl transferase inhibitors, COX-2 inhibitors, MMP inhibitors, mTOR inhibitors, antineoplastic antimetabolites, platin compounds, compounds decreasing the protein kinase activity and further anti-angiogenic compounds, gonadorelin agonists, anti-androgens, bengamides, bisphosphonates, antiproliferative antibodies and temozolomide (TEMODAL®).

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. A combination of the invention comprising an antineoplastic agent which is an aromatase inhibitor may particularly be useful for the treatment of hormone receptor positive breast tumors.

The term “antiestrogens” as used herein relates to compounds which antagonize the effect of estrogens at the estrogen receptor level. The term includes, but is not limited to tamoxifen, fulvestrant, raloxifene and raloxifene hydrochloride.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804).

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D.

The term “alkylating agents” as used herein includes, but is not limited to cyclophosphamide, ifosfamide and melphalan.

The term “histone deacetylase inhibitors” relates to compounds which inhibit the histone deacetylase and which possess antiproliferative activity.

The term “farnesyl transferase inhibitors” relates to compounds which inhibit the famesyl transferase and which possess antiproliferative activity.

The term “COX-2 inhibitors” relates to compounds which inhibit the cyclooxygenase type 2 enyzme (COX-2) and which possess anbproliferative activity such as celecoxib (Celebrex®), rofecoxib (Vioxx®) and lumiracoxib (COX189).

The term “MMP inhibitors” relates to compounds which inhibit the matrix metalloproteinase (MMP) and which possess antiproliferative activity.

The term “antineoplastic antimetabolites” includes, but is not limited to 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719.

The term “platin compounds” as used herein includes, but is not limited to carboplatin, cis-platin and oxaliplatin.

The term “compounds decreasing the protein kinase activity and further anti-angiogenic compounds” as used herein includes, but is not limited to compounds which decrease the activity of e.g. the Vascular Endothelial Growth Factor (VEGF), the Epidermal Growth Factor (EGF), c-Src, protein kinase C, Platelet-derived Growth Factor (PDGF), Bcr-Abl tyrosine kinase, c-kit, Flt-3 and Insulin-like Growth Factor I Receptor (IGF-IR) and Cyclin-dependent kinases (CDKs), and anti-angiogenic compounds having another mechanism of action than decreasing the protein kinase activity.

Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula I), WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58,1998, 3209-3214, and by J. Mordenti et al in Toxicologic Pathology, vol. 27, no. 1, pp 14-21, 1999; in WO 00/37502 and WO 94/10202; Angiostatin™, described by M. S. O'Reilly et al, Cell 79, 1994, 315-328; and Endostatin™, described by M. S. O'Reilly et al, Cell 88, 1997, 277-285; compounds which decrease the activity of EGF are especially compounds which inhibit the EGF receptor, especially the tyrosine kinase activity of the EGF receptor, and compounds binding to EGF, and are in particular those compounds generically and specifically disclosed in WO 97/02266 (describing compounds of formula IV), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980; compounds which decrease the activity of c-Src include, but are not limited to, compounds inhibiting the c-Src protein tyrosine kinase activity as defined below and to SH2 interaction inhibitors such as those disclosed in W097/07131 and W097/08193; compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706; compounds which decreases the activity of the protein kinase C are especially those staurosporine derivatives disclosed in EP 0 296 110 (pharmaceutical preparation described in WO 00/48571) which compounds are protein kinase C inhibitors; further specific compounds that decrease protein kinase activity and which may also be used in combination with the compounds of the present invention are Imatinib (Gleevec®/Glivec®), PKC412, Iressa™ (ZD1839), PKI166, PTK787, ZD6474, GW2016, CHIR-200131, CEP-7055/CEP-5214, CP-547632 and KRN-633; anti-angiogenic compounds having another mechanism of action than decreasing the protein kinase activity include, but are not limited to e.g. thalidomide (THALOMID), celecoxib (Celebrex), SU5416 and ZD6126.

The term “gonadorelin agonist” as used herein includes, but is not limited to abarelix, goserelin and goserelin acetate. Goserelin is disclosed in U.S. Pat. No. 4,100,274.

The term “anti-androgens” as used herein includes, but is not limited to bicalutamide (CASODEX™), which can be formulated, e.g. as disclosed in U.S. Pat. No. 4,636,505.

The term “bengamides” relates to bengamides and derivatives thereof having aniproliferative properties.

The term “bisphosphonates” as used herein includes, but is not limited to etridonic acid, clodronic acid, tiludronic acid, pamidronic acid, alendronic acid, ibandronic acid, risedronic acid and zoledronic acid.

The term “antiproliferative antibodies” as used herein includes, but is not limited to trastuzumab (Herceptin™), Trastuzumab-DM1, erlotinib (Tarceva™), bevacizumab (Avastin™), rituximab (Rituxan®), PRO64553 (anti-CD40) and 2C4 Antibody.

The structure of the active agents identified by code nos., generic or trade names may be taken from the actual edition of the standard compendium “The Merck IndeX” or from databases, e.g. Patents International (e.g. IMS World Publications). The compositions of the invention may be administered by any conventional route, in particular parenterally, for example in the form of injectable solutions or suspensions, enterally, e.g. orally, for example in the form of tablets or capsules, topically, e.g. in the form of lotions, gels, ointments or creams, or in a nasal or a suppository form. Pharmaceutical compositions comprising an agent of the invention in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance. Topical administration is e.g. to the skin. A further form of topical administration is to the eye.

The compounds of formula I may be administered in free form or in pharmaceutically acceptable salt form, e.g. as indicated above. Such salts may be prepared in conventional manner and exhibit the same order of activity as the free compounds.

The inhibition of ALK tyrosine kinase activity is measured using known methods, for example using the recombinant kinase domain of the ALK in analogy to the VEGF-R kinase assay described in J. Wood et al. Cancer Res. 60, 2178-2189 (2000). The table below reports the IC50 values for several compounds of the present invention. Each compound is tested twice, once each with two different preparations of ALK.

| compound | IC50 μM |
|---|---|
| Ex. 48 | 0.048 |
| Ex. 48 | 0.083 |
| Ex. 58 | 0.046 |
| Ex. 58 | 0.090 |
| Ex. 56 | 0.18 |
| Ex. 56 | 0.086 |

The compounds of formula I potently inhibit the growth of human NPM-ALK overexpressing murine BaF3 cells. The expression of NPM-ALK is achieved by transfecting the BaF3 cell line with an expression vector pCIneo™ (Promega Corp., Madison Wis., USA ) coding for NPM-ALK and subsequent selection of G418 resistant cells. Non-transfected BaF3 cells depend on IL-3 for cell survival. In contrast NPM-ALK expressing BaF3 cells (named BaF3-NPM-ALK) can proliferate in the absence of IL-3 because they obtain proliferative signal through NPM-ALK kinase. Putative inhibitors of the NPM-ALK kinase therefore abolish the growth signal and result in antiproliferaflve activity. The antiproliferative activity of putative inhibitors of the NPM-ALK kinase can however be overcome by addition of IL-3 which provides growth signals through an NPM-ALK independent mechanism. [for an analogous cell system using FLT3 kinase see E Weisberg et al. Cancer Cell; 1, 433-443 (2002). The inhibitory activity of the compounds of formula I is determined, briefly, as follows: BaF3-NPM-ALK cells (15 000/microtitre plate well) are transferred to 96-well microtitre plates. The test compounds [dissolved in dimethyl sulfoxide (DMSO)] are added in a series of concentrations (dilution series) in such a manner that the final concentration of DMSO is not greater than 1% (v/v). After the addition, the plates are incubated for two days during which the control cultures without test compound are able to undergo two cell-division cycles. The growth of the BaF3-NPM-ALK cells is measured by means of Yopro™ staining (T ldziorek et al. J. Immunol. Methods; 185:249-58 [1995]): 25 μl of lysis buffer consisting of 20 mM sodium citrate, pH 4.0, 26.8 mM sodium chloride, 0.4% NP40, 20 mM EDTA and 20 mM was added to each well. Cell lysis was completed within 60 min at room temperature and total amount of Yopro bound to DNA was determined by measurement using the Cytofluor II 96-well reader (PerSeptive Biosystems) with the following settings: Excitation (nm) 485/20 and Emission (nm) 530/25.

IC₅₀ values are determined by a computer-aided system using the formula:
ICr₅₀=[(ABSₜₑₛₜ−ABSₛₜₐᵣₜ)/(ABS_{control}−ABSₛₜₐᵣₜ)]×100.

The IC₅₀ value in those experiments is given as that concentration of the test compound in question that results in a cell count that is 50% lower than that obtained using the control without inhibitor. The compounds of formula I exhibit inhibitory activity with an IC₅₀ in the range from approximately 0.01 to 1 μM.

The antiproliferative action of the compounds of formula I can also be determined in the human KARPAS-299 lympoma cell line (described in WG Dirks et al. Int. J. Cancer 100, 49-56 (2002) using the same methodology described above for the BaF3-NPM-ALK cell line. The compounds of formula I exhibit inhibitory activity with an IC₅₀ in the range from approximately 0.01 to 1 μM.

The following compounds are tested in the cellular assays in the BaF3 cell lines and the KARPAS-299 cell line as described above:

| | BaF3 | BaF3 | |
|---|---|---|---|
| | NPM-ALK | NPM-ALK | KARPAS- |
| | with IL3 | without IL3 | 299 |
| | IC50 (μM) | IC50 (μM) | IC50 (μM) |
| Ex. 56 | 2.7 | 0.41 | 0.15 |
| Ex. 58 | 2.6 | 0.56 | 0.33 |
| Ex. 48 | 1.4 | 0.55 | 0.27 |

1. A method of treating or preventing a condition susceptible to treatment with an ALK inhibiting agent which comprises inhibiting ALK or a gene fusion thereof with a compound of formula I 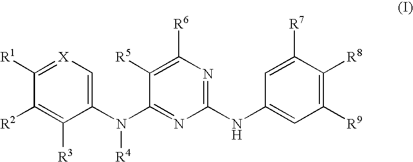
wherein
X is ═CR⁰— or ═N—;
each of R⁰, R¹, R², R³ and R⁴ independently is hydrogen; hydroxy; C₁-C₈alkyl; C₂-C₈alkenyl; C₃-C₈cycloalkyl; C₃-C₈cycloalkyl-C₁-C₈alkyl; hydroxyC₁-C₈alkyl; C₁-C₈alkoxyC₁-C₈alkyl; hydroxyC₁-C₈alkoxyC₁-C₈alkyl; arylC₁-C₈alkyl which optionally may be substituted on the ring by hydroxy, C₁-C₈alkoxy, carboxy or C₁-C₈alkoxycarbonyl;
or R³ and R⁴ form together with the nitrogen and carbon atoms to which they are attached a 5 to 10 membered heterocyclic ring and comprising additionally 1, 2 or 3 heteroatoms selected from N, O and S;
or each of R¹, R² and R³, independently, is halogen; halo-C₁-C₈alkyl; C₁-C₈alkoxy; halo-C₁-C₈alkoxy; hydroxyC₁-C₈alkoxy; C₁-C₈alkoxyC₁-C₈alkoxy; aryl; arylC₁-C₈alkoxy; heteroaryl; heteroaryl-C₁-C₄alkyl; 5 to 10 membered heterocyclic ring; nitro; carboxy; C₂-C₈alkoxycarbonyl; C₂-C₈alkylcarbonyl; —N(C₁-C₈alkyl)C(O) C₁-C₈alkyl; —N(R¹⁰)R¹¹; —CON(R¹⁰)R¹¹; —SO₂N(R¹⁰)R¹¹; or —C₁-C₄-alkylene-SO₂N(R¹⁰)R¹¹; wherein each of R¹⁰ and R¹¹ independently is hydrogen; hydroxy; C₁-C₈alkyl; C₂-C₈alkenyl; C₃-C₈cycloalkyl; C₃-C₈cycloalkyl-C₁-C₈alkyl; C₁-C₈alkoxyC₁-C₈alkyl; hydroxyC₁-C₈alkoxyC₁-C₈alkyl; hydroxyC₁-C₈alkyl; (C₁-C₈alkyl)-carbonyl; arylC₁-C₈alkyl which optionally may be substituted on the ring by hydroxy, C₁-C₈alkoxy, carboxy or C₂-C₈alkoxycarbonyl; or 5 to membered heterocyclic ring;
or R¹ and R² form together with the C-atoms to which they are attached aryl or a 5 to 10 membered heteroaryl residue comprising one or two heteroatoms selected from N, O and S; or
each of R⁵ and R⁶ independently is hydrogen; halogen; cyano; C₁-C₈alkyl; halo-C₁-C₈alkyl; C₂-C₈alkenyl; C₂-C₈alkynyl; C₃-C₈cycloalkyl; C₃-C₈cycloalkylC₁-C₈alkyl; C₅-C₁₀arylC₁-C₈alkyl;
each of R⁷, R⁸ and R⁹ is independently hydrogen; hydroxy; C₁-C₈alkyl; C₂-C₈alkenyl; halo-C₁-C₈alkyl; C₁-C₈alkoxy; C₃-C₈cycloalkyl; C₃-C₈cycloalkylC₁-C₈alkyl; arylC₁-C₈alkyl; —Y—R¹² wherein Y is a direct bond or O and R¹² is a substituted or unsubstituted 5, 6 or 7 membered heterocyclic ring comprising 1, 2 or 3 heteroatoms selected from N, O and S; carboxy; (C₁-C₈alkoxy)-carbonyl; —N(C₁₋₈alkyl)-CO—NR¹⁰R¹¹; —CONR¹⁰R¹¹; —N(R¹⁰)(R¹¹); —SO₂N(R¹⁰)R¹¹; R⁷ and R⁸ or R⁸ and R⁹, respectively form together with the carbon atoms to which they are attached, a 5 or 6 membered heteroaryl comprising 1, 2 or 3 heteroatoms selected from N, O and S; or a 5 or 6 membered carbocyclic ring.
in free form or salt form.

2. A method according to claim 1 wherein at most one of R¹, R² or R³ is —CON(R¹⁰)R¹¹; or —SO₂N(R¹⁰)R¹¹.

3. A method of claim 1 wherein the condition is a proliferative disease.

4. A method of claim 1 wherein a gene fusion containing ALK is inhibited.

5. A method for the treatment of a hematological or neoplastic disease comprising administering a compound of formula I 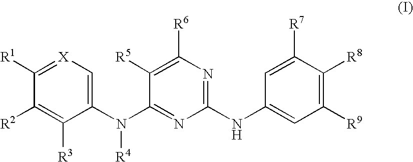
wherein
X is ═CR⁰— or ═N—;
each of R⁰, R¹, R², R³ and R⁴ independently is hydrogen; hydroxy; C₁-C₈alkyl; C₂-C₈alkenyl; C₃-C₈cycloalkyl; C₃-C₈cycloalkyl-C₁-C₈alkyl; hydroxyC₁-C₈alkyl; C₁-C₈alkoxyC₁-C₈alkyl; hydroxyC₁-C₈alkoxyC₁-C₈alkyl; arylC₁-C₈alkyl which optionally may be substituted on the ring by hydroxy, C₁-C₈alkoxy, carboxy or C₁-C₈alkoxycarbonyl;
or R³ and R⁴ form together with the nitrogen and carbon atoms to which they are attached a 5 to 10 membered heterocyclic ring and comprising additionally 1, 2 or 3 heteroatoms selected from N, O and S;
or each of R¹, R² and R³, independently, is halogen; halo-C₁-C₈alkyl; C₁-C₈alkoxy; halo-C₁-C₈alkoxy; hydroxyC₁-C₈alkoxy; C₁-C₈alkoxyC₁-C₈alkoxy; aryl; arylC₁-C₈alkoxy; heteroaryl; heteroaryl-C₁-C₄alkyl; 5 to 10 membered heterocyclic ring; nitro; carboxy; C₂-C₈alkoxycarbonyl; C₂-C₈alkylcarbonyl; —N(C₁-C₈alkyl)C(O) C₁-C₈alkyl; —N(R¹⁰)R¹¹; —CON(R¹⁰)R¹¹; —SO₂N(R¹⁰)R¹¹; or —C₁-C₄-alkylene-SO₂N(R¹⁰)R¹¹; wherein each of R¹⁰ and R¹¹ independently is hydrogen; hydroxy; C₁-C₈alkyl; C₂-C₈alkenyl; C₃-C₈cycloalkyl; C₃-C₈cycloallyl-C₁-C₈alkyl; C₁-C₈alkoxyC₁-C₈alkyl; hydroxyC₁-C₈alkoxyC₁-C₈alkyl; hydroxyC₁-C₈alkyl; (C₁-C₈alkyl)-carbonyl; arylC₁-C₈alkyl which optionally may be substituted on the ring by hydroxy, C₁-C₈alkoxy, carboxy or C₂-C₈alkoxycarbonyl; or 5 to membered heterocyclic ring;
or R¹ and R² form together with the C-atoms to which they are attached aryl or a 5 to 10 membered heteroaryl residue comprising one or two heteroatoms selected from N, O and S; or
each of R⁵ and R⁶ independently is hydrogen; halogen; cyano; C₁-C₈alkyl; halo-C₁-C₈alkyl; C₂-C₈alkenyl; C₂-C₈alkynyl; C₃-C₈cycloalkyl; C₃-C₈cycloalkylC_{1-C}₈alkyl; C₅-C₁₀arylC₁-C₈alkyl;
each of R⁷, R⁸ and R⁹ is independently hydrogen; hydroxy; C₁-C₈alkyl; C₂-C₈alkenyl; halo-C₁-C₈alkyl; C₁-C₈alkoxy; C₃-C₈cycloalkyl; C₃-C₈cycloalkylC₁-C₈alkyl; arylC₁-C₈alkyl; —Y—R¹² wherein Y is a direct bond or 0 and R¹² is a substituted or unsubstituted 5, 6 or 7 membered heterocyclic ring comprising 1, 2 or 3 heteroatoms selected from N, O and S; carboxy; (C₁-C₈alkoxy)-carbonyl; —N(C₁₋₈alkyl)-CO—NR¹⁰R¹¹; —CONR¹⁰R¹¹; —N(R¹⁰)(R¹¹); —SO₂N(R¹⁰)R¹¹; R⁷ and R⁸ or R⁸ and R9, respectively form together with the carbon atoms to which they are attached, a 5 or 6 membered heteroaryl comprising 1, 2 or 3 heteroatoms selected from N, O and S; or a 5 or 6 membered carbocyclic ring. in free form or salt form.

6. A method according to claim 5 wherein at most one of R¹, R² or R³ is —CON(R¹⁰)R¹¹; or —SO₂N(R¹⁰)R¹¹.

7. A method according to claim 5 wherein the condition is a proliferative disease.

48. A method according to claim 5 wherein a gene fusion containing ALK is inhibited.
